(19) United States
(12) Patent Application Publication
(10) Pub. No.: US 2009/0123409 A1
(43) Pub. Date: May 14, 2009

(54) ENHANCED SHAVING COMPOSITIONS

(76) Inventor: Milton D. Moore, Pearland, TX (US)

Correspondence Address: CONLEY ROSE, P.C.;David A. Rose, P. O. BOX 3267, HOUSTON, TX, 77253-3267, US

(21) Appl. No.: 12/263,860

(22) Filed: Nov. 3, 2008

Related U.S. Application Data
(60) Provisional application No. 60/984,573, filed on Nov. 1, 2007.

(51) Int. Cl. A61K 8/97; A61K 36/02; A61Q 9/02

(52) U.S. Cl. 424/73; 424/195.17

(57) ABSTRACT

A method for treating human skin comprising a shaving composition consisting of algae extract, willow bark extract, de-ionized water, propylene glycol, aloe vera, guar gum, diazolidinyl urea, and iodopropynyl butylcarbamate. The method further comprising applying the shaving composition to the skin prior to shaving the skin.

### CROSS-REFERENCE TO RELATED APPLICATIONS

This application claims the benefit under 35 U.S.C. § 119(e) of U.S. Provisional Patent Application No. 60/984,573, filed Nov. 1, 2007, the disclosure of which is incorporated herein by reference in their entirety for all purposes.

### STATEMENT REGARDING FEDERALLY SPONSORED RESEARCH OR DEVELOPMENT

Not applicable

### BACKGROUND

1. Field of the Invention

This invention relates generally to the field of shaving products. More specifically, the invention relates to enhanced shaving compositions.

2. Background of the Invention

For conventional shaving, a soap, gel, foaming gel, or foam is applied on the designated area before using the blade. The purpose of this application is to lubricate the region and make the shave comfortable.

Yet, there are some drawbacks for using regular soaps and foams. The hydration of hair is incomplete in some cases. Hair hydration or saturation is a means to reduce skin irritation, nicks, and cuts during shaving. Dehydration of the skin is another problem associated with regular soaps and foams. Certain lubricating components are hypotonic, resulting in dehydration of the epidermis. As such, creams and emollients are applied after shaving to overcome the irritation caused by the dehydration. The dehydration and then re-hydration of the skin is not an efficient procedure, and impacts skin elasticity, smoothness, suppleness, and resistance to irritants.

Certain surfactants and fillers in shaving compositions actually aggravate the aforementioned conditions resulting in a rough, cracked appearance of the skin. In extreme cases, the skin is comprised. It should also be noted that conventional soaps do not provide antimicrobial protection for skin during shaving operations. Additionally, shaving compositions are removed from the shaved region of skin with tap water. Tap water, comprising either a mineral rich water or artificially softened water further dries the skin. Salts and mineral salts affect the electrolyte balance of the epidermis and result in a loss of skin hydration.

Consequently, there is a need in the industry for enhanced shaving compositions that provide superior lubrication and hydration to the skin.

### BRIEF SUMMARY

Enhanced shaving compositions and methods are described herein. The methods and solutions incorporate the use of algae extracts for improved lubrication and hydration. Other aspects and features of the invention will be described in more detail below.

In one embodiment the hydrating shaving composition comprises: algae extract at a concentration of at least 0.5 wt %, willow bark extract a concentration ranging from about 0.01 wt % to about 0.25 wt %, de-ionized water at a concentration ranging from about 30 wt % to about 90 wt %, propylene glycol at a concentration ranging from about 12 wt % to about 55 wt %, aloe vera at a concentration ranging from about 0.5 wt % to about 1.5 wt %, guar gum at a concentration ranging from about 0.1 wt % to about 3 wt %, diazolidinyl urea at a concentration ranging from about 0.01 wt % to about 0.7 wt %, and iodopropynyl butylcarbamate at a concentration ranging from about 0.001 wt % about 0.1 wt %.

It should be appreciated by those skilled in the art that the conception and the specific embodiments disclosed may be readily utilized as a basis for modifying or designing other structures for carrying out the same purposes of the invention. It should also be realized by those skilled in the art that such equivalent constructions do not depart from the spirit and scope of the invention as set forth in the appended claims.

### DETAILED DESCRIPTION OF THE PREFERRED EMBODIMENTS

The skin composition of the invention provides better hydration of the hairs and lubrication of the skin than is achieved by conventional methods, such as using a shaving foam or soap in combination with water. As used herein, the skin composition may comprise a solution, gel, foam, liquid, crème, or emollient as understood by one skilled in the art for applying to the skin.

In one embodiment, the skin composition is suitable for use as a hydrating shave solution. Conventional methods tend to dry the skin and leave skin irritated a short time, after the shave. This enhanced hydration increases the ease with which a razor cuts through the hairs, reduces nicks and cuts, which makes the solution and system of the present invention well suited for those suffering from mild forms of razor rash, razor burn, or those having sensitive skin. This composition also reduces the chance of infection in the areas that are shaved wherein the skin is partially compromised by nicks due to personal error.

In further applications, the shaving composition may comprise a skin pre-treatment for application to the skin prior to applying a medical treatment thereto. Without wishing to be limited by theory, the hydration of the skin by the disclosed composition, may act to make the skin more permeable to trans-dermal medical applications. In certain instances, hydration of the skin by the disclosed composition may be beneficial for eczema, psoriasis, or anti-wrinkle treatment. Alternatively, the composition may be applied to the face prior to make-up application, in order to hydrate the skin, prevent pore clogging, or acne.

In an embodiment, a shaving composition comprises algae extract by weight percentage from about 0.05% to 8%, alternately from about 1% to about 3%, and preferably from about 1.5% to about 2.5%. The algae extract may be derived from any suitable freshwater algae species. In particular, examples of suitable algae species include without limitation, green algae, red algae, brown algae, or combinations thereof. Alternatively, the shaving composition may comprise seaweed, or marine algae extract. In an exemplary embodiment, marine algae extract may comprise a kelp extract such as alginate. In certain embodiments, the extract may comprise an extract from cyanobacteria, or “blue-green algae.”

Algae extract normalizes the skin's moisture content, and provides suppleness to the epidermis. Furthermore, the algae extract provides superior lubrication and hydration to the skin. Algae extract is a non-irritating and noncomedogenic. Accordingly, using the shaving compositions comprising algae extract provides improved means and rate of action for hydration and lubrication. In certain instances, it may be understood that this precludes the need for separate hydration after shaving.

The shaving composition is an aqueous solution such that the active ingredients are dissolved in a solvent. In certain embodiments, the solvent is water; alternatively, distilled water, and in preferred embodiments, the solvent is distilled, deionized water. Water comprises from about 30 wt % to about 90 wt %, alternately from about 40 wt % to about 80 wt, alternately from about 63 wt % to about 72 wt %. The solvent may be dispensed by an atomizer, pump spray, or propellant based fluid, gel, or foam dispenser, without limitation. In certain embodiments, the solvent comprises a water based gel or foam.

The shaving composition also comprises propylene glycol from about 12 wt % to about 60 wt %, alternatively from about 18 wt % to about 40 wt %, alternately from about 23% to about 30% by weight. In further embodiments, the shaving composition may comprise aloe vera, or aloe vera extract, at a concentration ranging from about 0.1 wt % to about 5 wt %, alternatively ranging from about 0.25 wt % to about 3 wt %, alternatively ranging from about 0.5 wt % to about 2 wt %.

Additionally, the shaving composition may comprise cationic polymers such as guar gum modified with hydroxyalkyl groups at concentration from about 0.1 wt % to about 3 wt %, preferably between 0.2 wt % and 2 wt %, and more preferably between 0.3 wt % and 1 wt %. In certain embodiments, the guar gum may comprise a commercially available product such as Jaguar® available from Rhodia. Further, the hydroxyalkyl groups may comprises alkyl groups with any number of carbon atoms. In a particular embodiment, the guar gum is modified with hydroxypropyl groups. Without being limited by theory, cationic polymers are believed to provide protection against disruptions of the cell surface by surfactants.

In additional embodiments, the shaving composition may comprise a low humidity humectant. The low humidity humectant may be at a concentration ranging from about 0.5 wt % to about 15 wt %; alternatively, from about 1 wt % to about 10 wt %, and preferably, from about 3 wt % to about 8 wt %. A low humidity humectant may comprise a commercially available compound, such as, without limitation, hydroxypropyl Bis-hydroxyethyldimonium chloride (Cola® moist 200). The shaving composition may further comprise a willow bark extract. The concentration of willow bark extract comprising about 0.01 wt % to about 3 wt %, preferably from about 0.05 wt % to about 1 wt %, and more preferably from about 0.1 wt % to about 0.25 wt %. Willow bark extract includes the compound salicylic acid; know to be an anti-inflammatory agent. Without limitation by any particular theory, the anti-inflammatory agent reduces adverse skin reactions to the shaving process. The solution may also comprise diazolidinyl urea in a concentration from about 0.01 wt % to about 3 wt %, preferably from 0.1 wt % to 0.7 wt %. Further, the solution may comprise iodopropynyl butylcarbamate at concentrations of at most 0.1 wt %, preferably between 0.001 wt % and 0.02 wt %. The combinations of diazolidinyl urea and iodopropynyl butylcarbamate are available as Germall® Plus or Liquid Germall® Plus, and are know to have antifungal and antibacterial properties.

In a preferred embodiment, the shaving composition further comprises an anti-oxidant agent. Examples of suitable anti-oxidant compounds include Vitamins A, C, D, E, beta-carotene, selenium, and zinc without limitation. Preferably, the anti-oxidant is Vitamin C. In certain embodiments, these antioxidants may have a concentration from about 0.01 wt % to about 10 wt %.

Other ingredients in the composition may include without limitation, preservatives, emulsifiers, and the like, without limitations. As understood by one skilled in the art, these compounds have beneficial properties within a shaving composition. In certain embodiments, these supplemental components may have a concentration from about 0.01 wt % to about 4 wt %.

In use, the area to be shaved is flooded with the composition of the invention. The disclosed compositions are preferably applied with an atomizer. The area is then shaved with any suitable shaving device. As used herein, shaving device refers to any device used to remove hair from the body such as without limitation, clippers, razors, straight razors, razor blades, disposable razors, multi-bladed razors, double-edge razors, and the like, without limitation.

The described properties comprising hydration, cell protecting, antimicrobial, anti-inflammatory of the said solution eliminate the need for any after-shave emollients and rinsing. To further illustrate various illustrative embodiments of the present invention, the following example is provided.

### EXAMPLE

An exemplary embodiment for a recipe for the disclosed shaving composition is shown in Table 1:

TABLE 1

| Hydroglide Plus | |
|---|---|
| Ingredients | % W/W |
| Di Water | 65.75 |
| Jaguar HP-8 | 0.5 |
| Propylene glycol | 25 |
| Cola ® moist 200 | 5 |
| Aloe Vera | 1 |
| Algae extract | 2 |
| Willow bark extract | 0.15 |
| Liquid Germall ® plus | 0.6 |
| Total | 100 |

While embodiments of the invention have been shown and described, modifications thereof can be made by one skilled in the art without departing from the spirit and teachings of the invention. The embodiments described and the examples provided herein are exemplary only, and are not intended to be limiting. Many variations and modifications of the invention disclosed herein are possible and are within the scope of the invention. Accordingly, the scope of protection is not limited by the description set out above, but is only limited by the claims, which follow, that scope including all equivalents of the subject matter of the claims.

We claim:

1. A shaving composition for shaving skin, comprising a de-ionized water solution, an algae extract, propylene glycol, aloe vera, guar gum, low humidity humectant, anti-inflammatory compound, diazolidinyl urea, and iodopropynyl butylcarbamate.

2. The shaving composition of claim 1 wherein the concentration of the algae extract is at least 0.5 wt %.

3. The shaving composition of claim 2, wherein the algae extract comprises at least one algae extract selected from the group consisting of aquatic algae, marine algae, cyanobacteria (blue-green) algae, and combinations thereof.

4. The shaving composition of claim 1 wherein the concentration of the deionized water solution is from about 30 wt % to about 90 wt %.

5. The shaving composition of claim 1 wherein concentration of the de-ionized water solution is from about 50 wt % to about 72 wt %.

4. The shaving composition of claim 1 wherein the concentration of the propylene glycol is from about 12 wt % to about 60 wt %.

5. The shaving composition of claim 1 wherein concentration of the aloe vera is from about 0.1 wt % to about 5 wt %.

6. The shaving composition of claim 1 wherein the concentration of the guar gum is from about 0.1 wt % to about 3 wt %.

7. The shaving composition of claim 7 wherein the guar gum further comprises hydroxyalkyl groups.

8. The shaving composition of claim 7 wherein the guar gum further comprises hydroxypropyl groups.

9. The shaving composition of claim 1 wherein the concentration of the low humidity humectant is from about 0.5 wt % to about 15 wt %

10. The shaving composition of claim 1 wherein the anti-inflammatory compound comprises a willow bark extract and has a concentration from about 0.01 wt % to about 0.25 wt %.

11. The shaving composition of claim 1 wherein the concentration of the diazolidinyl urea is from about 0.01 wt % to about 0.7 wt %

12. The shaving composition of claim 1 wherein the concentration of the iodopropynl butylcarbamates is from about 0.001 wt % to about 0.01 wt %

13. The shaving composition of claim 1 further comprising at least one supplemental component chosen from the group consisting of preservatives, emulsifiers, atomizers, and combinations thereof.

14. The shaving composition of claim 14, wherein the concentration of the at least one supplemental component is from about 0.1 wt % to about 4 wt %.

15. The shaving composition of claim 1 further comprising an antioxidant agent.

16. A skin-hydrating composition comprising:
at least 0.5 wt % algae extract;
from about 0.01 wt % to about 0.25 wt % willow bark; from about 30 wt % to about 90 wt % de-ionized water;
from about 12 wt % to about 55 wt % propylene glycol;
from about 0.5 wt % to about 1.5 wt % aloe vera;
about 0.1 wt % to about 3 wt % guar gum;
from about 0.01 wt % to about 0.7 wt % diazolidinyl urea; and
from about 0.001 wt % about 0.1 wt % iodopropynyl butylcarbamate.

17. A method of shaving a human skin area using a shaving device comprising:
applying an aqueous shaving composition comprising an algae extract, a de-ionized water solution, propylene glycol, aloe vera, guar gum, a low humidity humectant, willow bark extract, antioxidants, diazolidinyl urea, and iodopropynyl butylcarbamate to the human skin area; and
shaving the human skin area with a shaving device.

18. The method of claim 17 wherein further comprising atomizing the aqueous shaving composition.

19. A shaving composition, comprising a de-ionized water solution and an algae extract.

20. The shaving composition of claim 19, wherein the concentration of the algae extract is at least 0.5 wt % and said algae extract comprises at least one algae extract selected from the group consisting of aquatic algae, marine algae, cyanobacteria (blue-green) algae, and combinations thereof.
